(19) United States
(12) Patent Application Publication
(10) Pub. No.: US 2020/0085512 A1
(43) Pub. Date: Mar. 19, 2020

(54) TOOLS AND TECHNIQUES FOR IMAGE-GUIDED RESECTION

(71) Applicant: IMRIS, Inc., Minnetonka, MN (US)

(72) Inventors: Brendan Reimer, Eden Prairie, MN (US); Meir Dahan, St. Louis Park, MN (US); Gord Scarth, Eden Prairie, MN (US)

(21) Appl. No.: 16/610,355

(22) PCT Filed: May 8, 2018
(86) PCT No.: PCT/US2018/031678

§ 371 Date: Nov. 1, 2019

Related U.S. Application Data
(60) Provisional application No. 62/503,584, filed on May 9, 2017.

(51) Int. Cl. A61B 34/20; A61B 5/055

(52) U.S. Cl. CPC A61B 34/20; A61B 18/1492; A61B 5/055

(57) ABSTRACT

A region within a body of an imaging subject can be imaged to identify a tumor locus in a three dimensional coordinate system. An opening in the body of the imaging subject can be formed to provide an access location. Using an actuator, a surgical tool can be guided to traverse the access location to access the tumor locus, the surgical tool guided along a specified trajectory in the three dimensional coordinate system by the actuator and configured to resect and remove a first portion of the tumor within the tumor locus.

### CLAIM OF PRIORITY

This patent application claims the benefit of priority of Reimer et al., U.S. Provisional Patent Application Ser. No. 62/503,584, titled “SURGICAL TOOL AND TECHNIQUES FOR MINIMALLY-INVASIVE IMAGE-GUIDED TUMOR RESECTION,” filed on May 9, 2017 (Attorney Docket No. 4336.002PV4), which is hereby incorporated by reference herein in its entirety.

### BACKGROUND

A surgical procedure can include use of a stereotactic instrument in order to guide a surgical device such as a needle to a site of a tumor or other lesion. In one approach, imaging of a subject (e.g., a patient) can be performed, such as prior to securely fixing a stereotactic instrument such as a frame to the subject. The pre-operative imaging can be used to develop a surgery plan. The plan can then be executed manually by the surgeon using the stereotactic instrument to position the surgical device according to the plan. In one approach, the surgical instrument can be a laser ablation device, and the plan can include guiding the laser ablation device to a tumor site to thermally ablate the tumor. Post-operatively, further imaging can be performed such as to assess the efficacy of the procedure.

### OVERVIEW

The present inventors have recognized, among other things, that large tumors or other lesions can be difficult to treat using generally-available stereotactically-guided laser ablation techniques. As a volume or cross section of ablated tissue increases, difficulties can arise in controlling an amount of thermal damage inflicted by the laser ablation tool, particularly at a periphery or margin of a tumor locus. Accordingly, the present inventors have recognized, among other things, that other approaches can be used to treat or remove a tumor. For example, imaging can be used to develop a specified trajectory to be followed by a surgical tool such as can be used to mechanically resect or otherwise remove a portion of a tumor. In an example, a remaining or other portion of the tumor can be treated such as using an ablation tool. In another example, a portion or an entirety of the tumor can be treated using ablation, and a surgical tool can be used to remove ablated tumor or other damaged or dead tissue after ablation, such as to inhibit or suppress swelling or edema. A cannula can be used, such as to provide a path for one or more tools to traverse one or more anatomical structures such as a bony structure. In an example, the bony structure can include a cranium. One or more surgical tools used to perform resection or ablation can be manipulated, such as guided along a specified trajectory, using an actuator. In an example, the actuator can include multiple degrees of freedom, such as comprising a surgical robot or other apparatus to facilitate at least partially automating or assisting the guidance of the surgical tool along the specified trajectory. Imaging can be performed during or after resection or removal of a portion of the tumor, such as to assess progress in tumor removal. For example, intra-operative imaging can be performed to determine whether to perform further mechanical resection, or to determine an ablation therapy protocol. After ablation, imaging can be used to assess an effectiveness of a delivered ablation therapy. In an example, ablation therapy can be delivered, and mechanical resection can be performed after ablation, such as to suppress or inhibit one or more of edema or swelling.

In an example, a region within a body of an imaging subject can be imaged to identify a tumor locus in a three dimensional coordinate system. An opening in the body of the imaging subject can be formed to provide an access location. Using an actuator, a surgical tool can be guided to traverse the access location to access the tumor locus, the surgical tool guided along a specified trajectory in the three dimensional coordinate system by the actuator and configured to resect and remove a first portion of the tumor within the tumor locus. Such resection can be performed one or more of before or after other activities such as ablation.

This overview is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

FIG. 1A through 1E illustrate generally a series of examples, such as can include one or more of apparatus or techniques for minimally-invasive resection of at least a portion of a tumor within an imaging subject.

FIGS. 2A through 2F illustrate generally a series of examples, such as can include one or more of apparatus or techniques for minimally-invasive resection of at least a portion of a tumor within a cranium of an imaging subject.

FIGS. 3A through 3C illustrate generally a series of examples, such as can include one or more of apparatus or techniques for minimally-invasive resection of at least a portion of a tumor, including or using a cannula.

FIG. 4 illustrates generally an illustrative example of at least a portion of a surgical device, such as can include a surgical tool or cannula.

FIGS. 5A and 5B illustrate generally examples of at least a portion of a surgical device, such as can include a surgical tool or cannula having laterally-facing ports.

FIGS. 6A and 6B illustrate generally examples of at least a portion of a surgical device, such as can include a surgical tool defining a lumen, where a distally-extending member can be deployed through the lumen and can extend in a radial direction when protruding beyond a distally-located opening of lumen.

FIG. 7 illustrates generally an example of at least a portion of a surgical device, such as can include a surgical tool defining a lumen, where a distally-extending shear or retractor can be deployed within the lumen.

### DETAILED DESCRIPTION

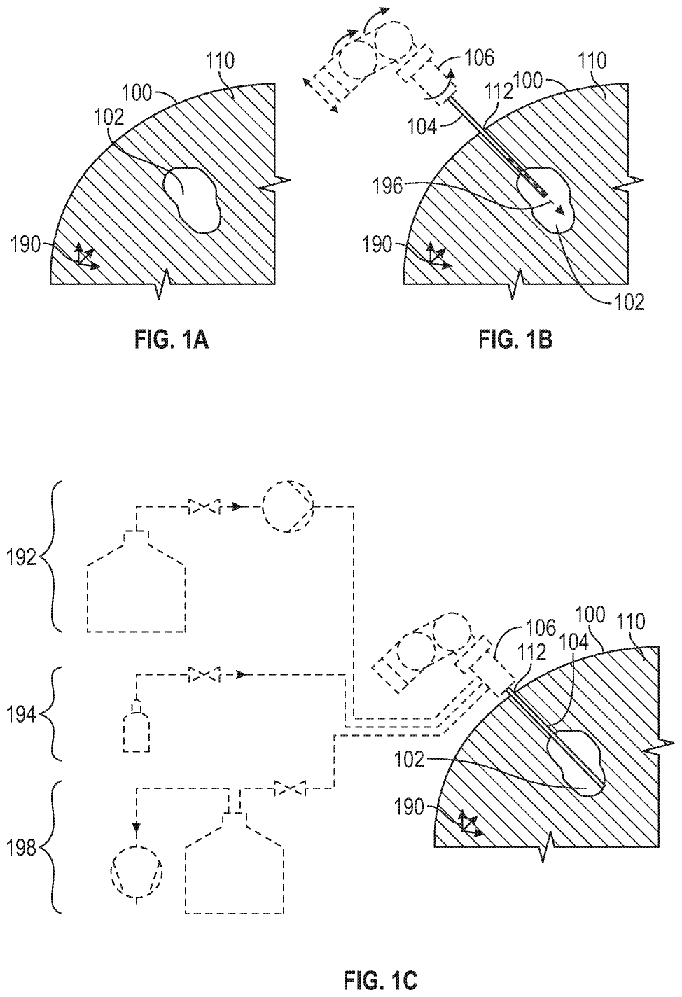

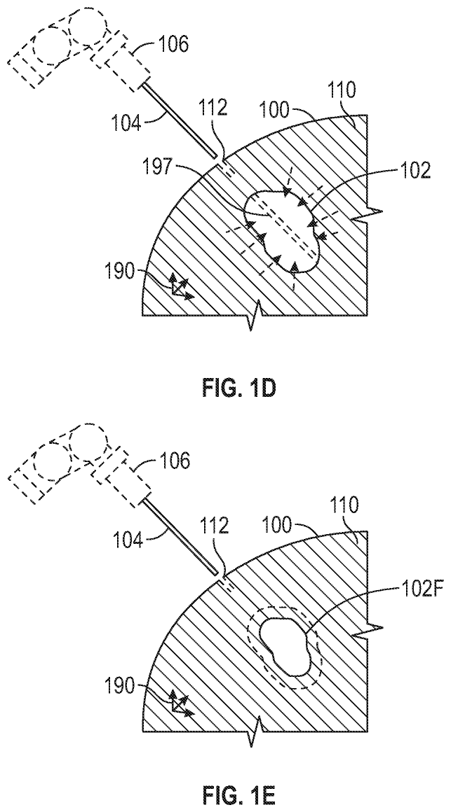

FIG. 1A through 1E illustrate generally a series of examples, such as can include one or more of apparatus or techniques for minimally-invasive resection of at least a portion of a tumor within an imaging subject. FIG. 1A illustrates generally an imaging subject 100. A tumor 102 location can be identified within an imaging region 110 within the imaging subject. The imaging can include one or more of computed tomography (CT) or nuclear magnetic resonance imaging (MRI), such as can include contrast-enhanced CT or MRI, or one or more other imaging modalities. The location of the tumor can be defined with respect to a three-dimensional coordinate system 190.

FIG. 1B illustrates generally one or more of apparatus or techniques such as can include using an actuator 106 to guide a surgical tool 104 through an access location 112 on a surface of the imaging subject 100, such as along a trajectory 196 specified in relation to the three-dimension coordinate system 190. The access location 112 can be determined using the imaging information obtained as mentioned above in relation to FIG. 1A. A penetration in the imaging subject to provide the access location 112 can be formed such as using a bur or drill (in the case of accessing the tumor 102 within a bony structure), or using a cannula or trocar having a distal tip portion configured to pierce the skin or other tissue of the imaging subject 100.

The actuator can include an electrically-actuated or mechanically-actuated positioners to provide multiple degrees of freedom, such as can include linear translation in one or more axes or rotation in one or more axes, as shown generally by the arrows located nearby the actuator 106 in FIG. 1B. In an illustrative example, general movement can be provided using six degrees of freedom. Such general movement can include one or more of positioning of a surgical tool 104 or cannula (as discussed below) within tissue, or positioning surgical tool 104 relative to a cannula or other device (e.g., for manipulation of the surgical tool 104 within a cannula, or for manipulation of other elongate members within the surgical tool 104). The actuator can be coupled to a controller, such as can include a processor circuit and a processor-readable medium including instructions that cause the processor circuit to determine an appropriate series of commands to move individual positioners comprising the actuator.

Such control can be executed according to a predetermined program or at least in part in response to user input, such as can include a commanded input from a user directing the motion. Other actuation apparatus or techniques can be used, such as a stereotactic frame including a fixation device anchored to the imaging subject 100, to perform the surgical techniques described herein. Use of the actuator 106 can allow a precise trajectory 190 to be planned and then followed during surgery (or revised during surgery using further imaging), such as to position the surgical tool 104 within or nearby the identified tumor 102 location.

Positioning of the surgical tool 105 using the actuator 106 can be referred to generally as a stereotactic technique, even though such a technique need not require use of a physical stereotactic frame coupled between the imaging subject 100 and the surgical tool 104. For example, the actuator can include a surgical robot anchored to a surgical operating table or platform, and the surgical operating table can include a fixation device to anchor the imaging subject to the table or platform. The actuator 106 and surgical tool 104 can be compatible with various imaging modalities such as CT or MRI, such as to facilitate intra-operative imaging during tumor identification or resection, or in support of (e.g., during) other diagnosis or treatment such as electrocautery or ablation.

FIG. 1C illustrates generally an example where the surgical tool 104 is configured to penetrate through at least a portion of the tumor 102, such as during or after positioning by the actuator 106. For example, the surgical tool 104 can be positioned along the trajectory 196 shown in FIG. 1B including accessing the tumor 102 through the access location 112. The surgical tool 104 can be configured to resect a portion of the tumor 102, such as using aspiration or other techniques. Various facilities can be coupled to the surgical tool 104 or actuator 104, such as can include a vacuum source 198, such as to facilitate aspiration of tissue (e.g., tumor 102 tissue) through a lumen defined by the surgical tool 104.

A gas source 194 can be provided, such as to assist in maintaining a pressure equilibrium between the region 110 within the imaging subject 100 and a region surrounding the imaging subject. For example, as discussed other examples described herein, leakage of fluid such as cerebrospinal fluid (CSF) can be inhibited or prevented, such as at least in part by managing (e.g., limiting) a pressure differential between the region surrounding the imaging subject 100 and a region 110 within the imaging subject, such as nearby a distal tip portion of the surgical tool 104. Elsewhere, one or more seals or other features can be included, such as to avoid gas or liquid leakage from the access location 112, such as when the access location 112 is through the cranium or dura. In an example, the surgical tool 104 can be configured to provide irrigation, such as in one or more of an axial or radial direction. Such irrigation can include saline provided by an irrigation source 192. The surgical tool can be configured to perform other techniques in addition or instead of those mentioned above, such as can include electrocautery or tissue resection using a distally-extending member deployed through or along the surgical tool 104 (e.g., a mechanical cutter such as a shear as shown and described in relation to FIG. 7).

FIG. 1D illustrates generally an example that can include removing the surgical tool 104 using the actuator 106 from the access location 112. For example, the surgical tool 104 can be used to aspirate a portion of the tumor 102, such as removing tissue using the assistance of a vacuum source as mentioned above or otherwise “coring” a portion 197 of the tumor 102. In an example where the tumor 102 is not entirely rigid or fibrotic, the remaining tumor 102 tissue can relax into a void formed by removed portion 197 of the tumor 102. In an example, such relaxation can be facilitated by application of vacuum to the surgical tool 104 while the tool is located within or nearby the tumor 102, such as may be enhanced or otherwise rendered more effective by use of a seal at or nearby the access location 112.

FIG. 1E illustrates generally an example such as can include a reduced tumor 102F, such as after relaxation as mentioned in relation to FIG. 1D. The surgical tool 104 can be re-inserted into the region 110 within the patient, such as using the existing access location 112 along a trajectory similar or identical to a previous trajectory, such as to perform another aspiration or coring operation or to otherwise resect or remove another portion of the reduced tumor 102F. In an example, the tool 104 can include a distally-located tip region that can be manipulated independently of other portions of the tool 104, such as to facilitate movement or access to regions within a tumor locus other than those located axially along the specified trajectory. For example, such distal tip movement can permit access to a three dimensional volume nearby a distal tip of the surgical tool 104.

In an example, an imaging operation can be performed with the surgical tool 104 partially retracted from the reduced tumor 102F or with the surgical tool 104 entirely removed from the region 110 within the imaging subject 100. As mentioned in relation to other examples herein, other surgical devices or tools can be used before or after the surgical tool 104 is used. For example, the surgical tool 104 can be removed entirely, and a laser ablation tool can be used to further treat (e.g., ablate) the reduced tumor 102F.

In another example, ablation can be performed, and the surgical tool 104 can be used after ablation to resect ablated portions of a tumor, such as to suppress or inhibit one or more of edema or swelling. For example, an ablation technique can be used to treat a tumor in a minimally-invasive manner. In an illustrative example, such a procedure can include using an ablation tool that is about 1 to about 2 millimeters (mm) in diameter to ablate a tissue volume on the order of about 10 cubic centimeters (cc) or larger, such as can include a radius of ablation from the ablation tool of about 2 centimeters (cm). To suppress or inhibit edema or swelling, the surgical tool 104 can then be used to remove at least a portion of a tumor after ablation.

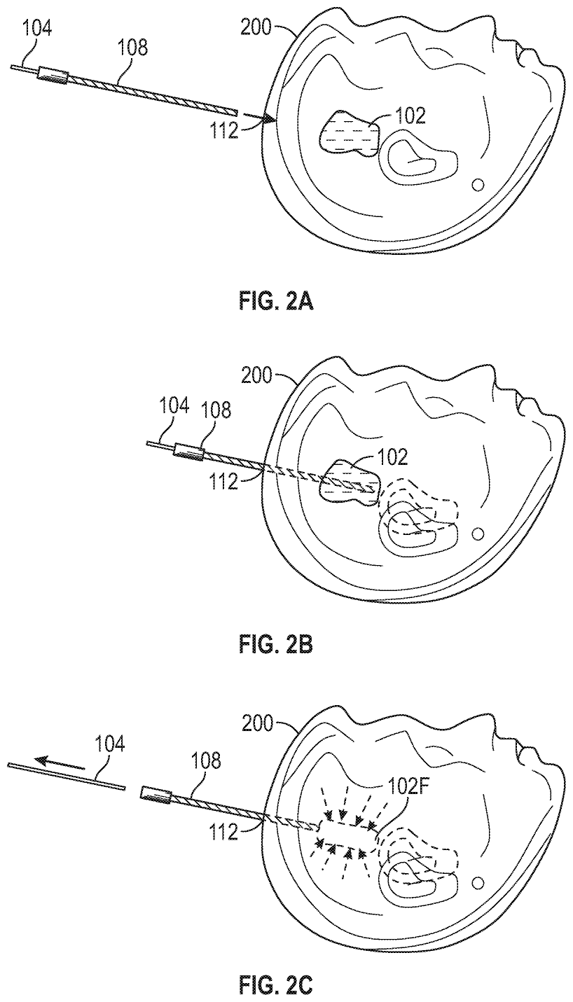

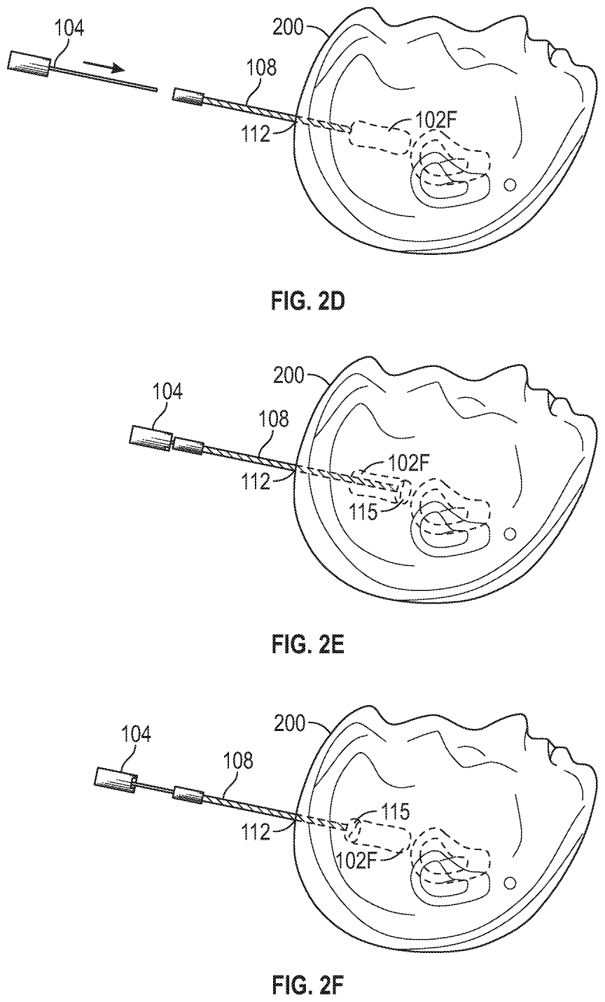

FIGS. 2A through 2F illustrate generally a series of examples, such as can include one or more of apparatus or techniques for minimally-invasive resection of at least a portion of a tumor within a cranium of an imaging subject. FIG. 2A illustrates generally an example that can include identifying a tumor 102 location, such as within a cranium 200 of an imaging subject. The identified tumor 102 location can be used to plan a trajectory for a minimally-invasive treatment, such as can include identifying an access location 112, and traversing the access location using a cannula 108. The cannula 108 can define a lumen such as through which one or more surgical tools such as a surgical tool 104 can be passed to access the tumor 102 location. Such positioning of the cannula 108 or surgical tool 104 can be facilitated by an actuator configured to follow a precisely-identified trajectory as mentioned in relation to other examples described herein.

FIG. 2B illustrates generally an example where the cannula 108 and the surgical tool 104 have been positioned to permit access to the tumor 102 location by the surgical tool 104, such as by traversing the access location 112. The surgical tool 104 can be configured to perform various techniques, such as one or more of tissue irrigation, tissue aspiration or resection, or electrocautery, for example. Tissue resection can be performed in a direction extending axially along a longitudinal axis of the surgical tool 104, or even radially, such as facilitated by one or more members that can travel along or through the surgical tool 104. FIG. 2C illustrates generally an example where the surgical tool 104 can be removed, such as including removing a portion of the tumor 102 to provide a reduced tumor 102F. The cannula 108 can remain in position, traversing the access location 112, such as to permit re-insertion or re-positioning of the surgical tool 104 or to permit access to the reduced tumor for further mechanical removal (e.g., resection) of tumor 102F tissue, or to facilitate other diagnosis or treatment. For example, the cannula 108 can be compatible with one or more imaging modalities such as CT or MRI. Imaging during or after removal of the surgical tool 104 can be used to plan further tissue removal or other treatment such as ablation. In another example, an ablation treatment can be delivered, and the surgical tool 104 can then be used to core out or otherwise resect tumor tissue, such as to suppress a tendency for swelling or edema development.

FIG. 2D illustrates generally an example that can include accessing the reduced tumor 102F location, such as using an ablation tool 114 (e.g., a laser ablation tool). A trajectory of the ablation tool 114 can be similar to the trajectory used by the surgical tool 104 for prior tissue removal. Such a trajectory can be planned in a three dimensional coordinate system such as using intra-operative imaging after the tissue removal performed using the surgical tool 104. FIG. 2E illustrates generally an example, that can include ablating a portion of the reduced tumor 102F. For example, a region 115 of thermal ablation can extend away from the ablation tool 114, such as one or more of axially or radially.

The present inventors have recognized, among other things, that a size and shape of the region 115 can inhibit ablation of an entirety of a tumor. Accordingly, a combination of mechanical tissue removal (such as using the surgical tool 104) and ablation can be used, such as including reducing the tumor using mechanical tissue removal, to a locus then treatable using ablation. In the example of FIG. 2E, an entirety of a cross-section of the reduced tumor 102F can be treated using ablation. FIG. 2F illustrates generally that the region 115 of ablation treatment can be swept along the reduced tumor 102F locus, such as by repositioning the ablation tool 114 within the cannula 108. In this manner, a tumor that might not otherwise be effectively treated using ablation can be treated using a combination of mechanical tumor tissue removal and ablation. As mentioned above, mechanical resection of tumor tissue can be performed one or more of before or after ablation.

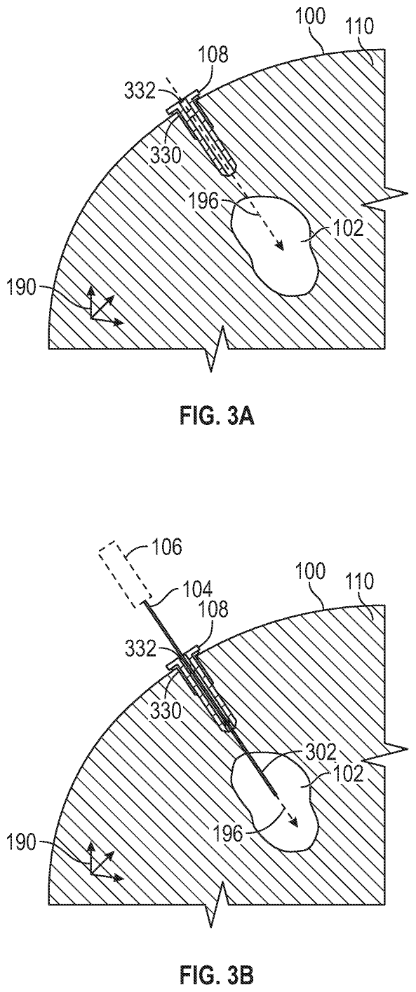

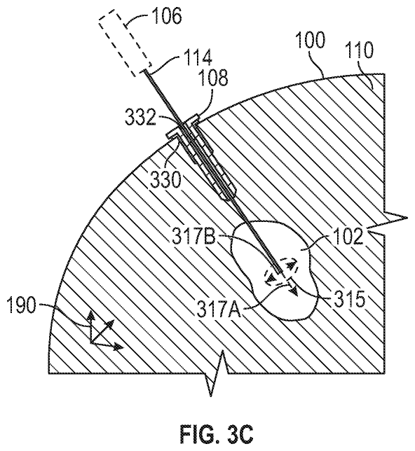

FIGS. 3A through 3C illustrate generally a series of examples, such as can include one or more of apparatus or techniques for minimally-invasive resection of at least a portion of a tumor, including or using a cannula 108. As mentioned in relation to other examples herein, a cannula 108 can be guided to traverse an access location, such as using an actuator according to a specified trajectory 196 in a three dimensional coordinate system 190. The specified trajectory can be determined using one or more of pre-operative or intra-operative imaging. As mentioned above, such positioning can be referred to as “stereotactic” even though such positioning need not require use of a generally-available manual stereotactic frame, because actuation can be one or more of machine-aided, machine-guided, or manually controlled.

FIG. 3A illustrates generally an illustrative example of cannula 108 that can include an interior seal 332. Such a seal can include a flexible or rigid material such as configured to provide a membrane or plug. For example, the seal can include a flexible elastic material such as silicone, such as can be pierced or otherwise penetrated by one or more other surgical tools (e.g., such as providing a septum). The seal can include a pre-formed aperture or slot. The interior seal 332 can be configured to suppress passage of gas or liquid such as during insertion or manipulation of one or more other surgical tools or before or after such insertion or manipulation. In this manner, leakage of fluid or gas from the region 110 within an imaging subject 100 through the cannula 108 can be suppressed or inhibited.

In an example, the interior seal 332 can include a balloon structure, for example, such as in the shape of a torus. The balloon structure can include a controlled inflation level, such as to provide a seal that forms to a surrounding area (such as between the surgical tool 104 and the cannula 108, or between the surgical tool 194 and surrounding anatomy (e.g., dura or inner cranium in the example of brain surgery) where the cannula 108 is not used or beyond the extent of the cannula 108.

The cannula 108 can include other features, such as an exterior seal 330. Such an exterior seal can include a rigid or flexible material, such as can include ribs or other features such as to suppress or inhibit leakage of gas or liquid in an annular region between the cannula 108 and tissue of the imaging subject 100. In another example, the exterior seal 330 can include one or more of a ring or torus, such as located between a flanged portion of the cannula 108 and an exterior surface of the imaging subject 100, As in the example of an interior seal 332 mentioned above, the ring or torus can include a balloon structure, such as inflatable to conform to surrounding structures (e.g., forming to the cranium in the example of brain surgery).

In another example, the exterior seal 330 can include a combination of features, such as located under a flange or shoulder of the cannula 108 and along the exterior wall of at least a portion of the cannula 108. As mentioned in relation to other examples described herein, suppression of leakage of gas or liquid across the seals 330 or 332 can be assisted or established at least in part by managing a pressure differential between the region 110 within the imaging subject 100 as compared to a region outside the region 110.

FIG. 3B illustrates generally an example that can include traversing an access location using a surgical tool 104, through a lumen defined by the cannula 108, along the specified trajectory 196, to provide access to the tumor 102 locus at a region 110 within an imaging subject 110. A portion 302 of the tumor 102 can be aspirated, such as through a lumen defined by the surgical tool 104. As mentioned in relation to other examples herein, the surgical tool 104 can be positioned using an actuator 106 according the specified trajectory 196 within the three dimensional coordinate system 196.

FIG. 3C illustrates generally an example that can include traversing an access location using an ablation tool 104, through the lumen defined by the cannula 108, along the specified trajectory 196, to provide access to the tumor 102 locus. The ablation tool can be configured to thermally ablate a region 315, such as extending in one or more of an axial direction 317A or a radial direction 317B. The ablation tool can include a laser ablation tool having an axially-directed output or a radially-directed output, such as having a capability to direct radiation in a particular azimuthal region radially.

In an illustrative example, imaging can be performed such as before ablation to determine an appropriate ablation program, and the ablation program can include positioning the ablation tool 114 along the specified trajectory using the actuator 106 and rotating the ablation tool to direct ablation to particular regions, also using the actuator 106. In another illustrative example, imaging can be performed after ablation to assess whether further ablation is necessary or to determine a revised trajectory. One or more of the cannula 108, the surgical tool 104, or the laser ablation tool 114 can be compatible with one or more imaging techniques, such as to permit intra-operative imaging. For example, the actuator 106 can be one or more of compatible with nuclear magnetic resonance imaging apparatus or computed tomography apparatus. In this manner, confirmation of progress in treating or resecting a tumor can be provided by near-real-time or real-time imaging. In an example, imaging information can be obtained during various stages of treatment, such as to guide resection and assess progress, or to guide ablation or assess ablation effectiveness. In an example, as mentioned above, a tumor can relax or can be encouraged to fill a void formed by resected tissue, such as to facilitate ablation or further resection, or tumor tissue can be removed after ablation to suppress or inhibit swelling or edema. Intra-operative imaging can be used to assess a degree of relaxation or to determine a revised trajectory or other protocol for further treatment.

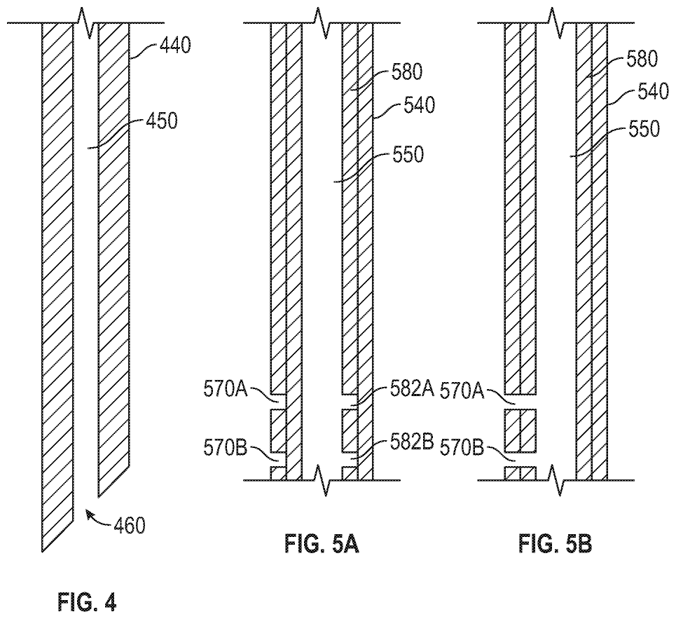

FIG. 4 illustrates generally an illustrative example of at least a portion of a surgical device 440, such as can include a surgical tool or cannula. The tool can define a lumen 450, such as through which other instruments or tools can be deployed, or through which tissue can be removed or irrigation delivered. A distal region 460 can include features such as a tapered end or other features such as to facilitate engaging or piercing tissue, such as shown. In an illustrative example, the device 440 can include stainless steel, polyurethane, or one or more other materials such as specified for physical characteristics e.g., rigidity or flexibility) and biocompatibility.

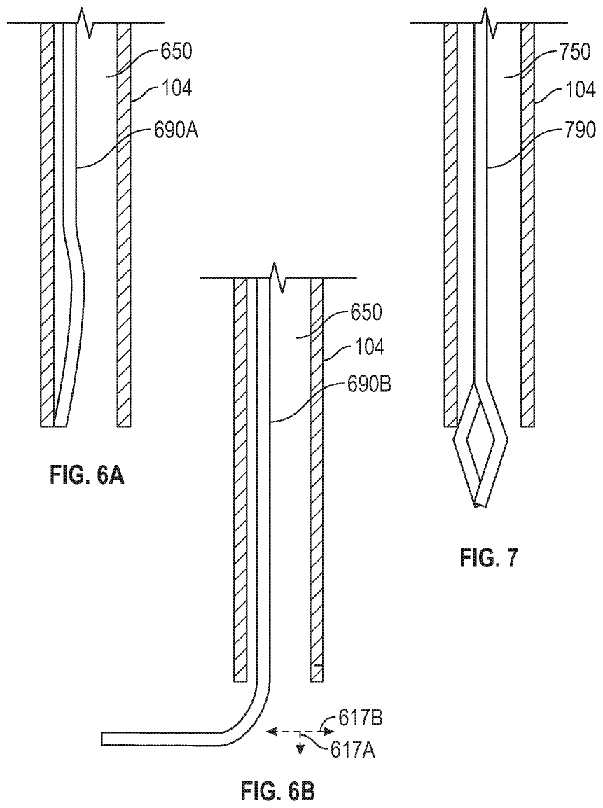

FIGS. 5A and 5B illustrate generally examples of at least a portion of a surgical device 540, such as can include a surgical tool or cannula having laterally-facing ports 570A and 570B. For example, such ports can be opened to a lumen 550 defined by the device 540, such as by using a sleeve 580. In the examples of FIGS. 5A and 5B, the sleeve can be repositioned to align apertures in the sleeve 580 with the ports 570A and 580B. In another example, the sleeve can be retracted or otherwise repositioned such as to expose the ports 570A or 570B to the lumen 550. While the examples of FIG. 4, FIG. 5A, FIG. 5B, FIG. 6A, FIG. 6B and FIG. 7 illustrate generally a single lumen, other configurations are possible, such as can include multiple lumen regions that can be concentric or non-concentric.

FIGS. 6A and 6B illustrate generally examples of at least a portion of a. surgical device, such as can include a surgical tool 104 defining a lumen 650, where a distally-extending member can be deployed through the lumen in an axial direction 617A and can extend in a radial direction 617B when protruding beyond a distally-located opening of lumen. Such a distally-extending member can include a shape-memory material, such as configured to remain in a generally-axially-extending configuration 690A when constrained by the tool 104, and extending radially in a deployed configuration 690B. For example, such a distally-extending member can be configured to manipulate or cut tissue, to provide irrigation, or to perform other operations such as provide one or more electrocautery electrodes. In an example, a wall of the tool 104 can provide one or more electrocautery electrodes. The distally-extending member can be positioned independently of the tool 104. For example, the tool 104 can be guided along a specified trajectory in a three-dimensional coordinate system by an actuator, and the distally-extending member can be positioned separately.

FIG. 7 illustrates generally an example of at least a portion of a surgical device, such as can include a surgical tool 104 defining a lumen 750, where a distally-extending shear or retractor 790 can be deployed within the lumen. Such a shear or retractor 790 can include one or more blades, such as one or more blades movable with respect to other portions of the shear or retractor 790, such as to assist in manipulating or cutting tissue. Such tissue can be irrigated such as using irrigation provided via the lumen 750. Tissue to be removed can be aspirated such as via the lumen 750 or using the shear or retractor 790 to engage or grip such tissue. In an example, the shear or retractor 790 can be positioned either manually by a user or using an actuator in an automated or semi-automated manner, such as independently of the positioning of the tool 104.

### VARIOUS NOTES & EXAMPLES

Each of these non-limiting examples can stand on its own, or can be combined in various permutations or combinations with one or more of the other examples.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C. F. R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1. A surgical system, comprising:
a surgical tool configured to resect a first portion of a tumor including traversing a lumen defined by a cannula to access a tumor locus, the tumor locus located within a body of an imaging subject;
an actuator configured to position the surgical tool according to a three dimensional coordinate system, the positioning including using information obtained from imaging a region within the imaging subject to identify the tumor locus in the three dimensional coordinate system;
wherein the surgical tool and the actuator are compatible with an imaging system and operable within or nearby the imaging system; and
wherein the imaging system comprises a computed tomography (CT) imaging system.

2. The surgical system of claim 1, comprising the cannula;
wherein the actuator is configured to position the cannula according to the three dimensional coordinate system, the positioning including using information obtained from imaging a region within the imaging subject to identify the tumor locus in the three dimensional coordinate system.

3. The surgical system of claim 2, comprising a laser ablation tool configured to provide laser radiation to thermally ablate a second portion of the tumor including traversing the lumen defined by the cannula to access the tumor locus; and
wherein the actuator is configured to position the laser ablation tool according to the three dimensional coordinate system, the positioning including using information obtained from imaging a region within the imaging subject to identify the tumor locus in the three dimensional coordinate system; and
wherein the laser ablation tool is compatible with the imaging system and operable within or nearby the imaging system.

4-7. (canceled)

8. The surgical system of claim 1, wherein the surgical tool is configured to:
cut tissue at a distally-located portion of the surgical tool; and
aspirate tissue through a lumen defined by the surgical tool.

9. The surgical system of claim 1, wherein the surgical tool is configured to deliver irrigation to a distally-located portion of the surgical tool.

10. The surgical system of claim 1, wherein the surgical tool is configured to cauterize tissue at a distally-located portion of the surgical tool.

11. The surgical system of claim 1, wherein the surgical tool is configured to cut tissue in a direction extending axially from a distally-located portion of the surgical tool.

12. The surgical system of claim 1, wherein the surgical tool is configured to cut tissue in a direction extending radially from a distally-located portion of the surgical tool.

13. The surgical system of claim 1, wherein the surgical tool is configured to access the tumor locus including traversing a bony structure.

14. The surgical system of claim 13, wherein the bony structure comprises a cranium.

15. The surgical system of claim 14,
wherein one or more of the cannula or the surgical tool are configured to inhibit or suppress leakage of cerebrospinal fluid (CSF) during a surgical procedure.

16. The surgical system of claim 15, wherein the cannula or surgical tool include a seal to inhibit or suppress the leakage.

17. The surgical system of claim 16, wherein the seal includes an inflatable structure.

18-22. (canceled)

23. A surgical system, comprising:
a cannula,
a surgical tool configured to resect a first portion of a tumor within a tumor locus located within a bony structure of subject including traversing a lumen defined by the cannula to access the tumor locus;
a laser ablation tool configured to provide laser radiation to thermally ablate a second portion of the tumor including traversing the lumen defined by the cannula to access the tumor locus; and
an actuator configured to stereotactically position the cannula, the surgical tool, and the laser ablation tool according to a three dimensional coordinate system, the stereotactic positioning including using information obtained from imaging a region within the bony structure of the subject to identify a tumor locus in the three dimensional coordinate system;
wherein the cannula, the surgical tool, the laser ablation tool, and the actuator are compatible with a nuclear magnetic resonance imaging system and operable within a magnetic field established by the nuclear magnetic resonance imaging system.

24. The surgical system of claim 23, wherein the surgical tool is configured to:
deliver irrigation to a distally-located portion of the surgical tool;
cut tissue a distally-located portion of the surgical tool; and
aspirate tissue through a lumen defined by the surgical tool.

25. The surgical system of claim 23, wherein the surgical tool is configured to cut tissue in a direction extending axially from a distally-located portion of the surgical tool.

26. The surgical system of claim 23, further comprising the nuclear magnetic resonance imaging system.

27. The surgical system of claim 23, wherein the bony structure comprises a cranium.

28. The surgical system of claim 23, wherein the first and second portions of the tumor overlap.

29-70. (canceled)

71. A surgical system, comprising:
a cannula defining a lumen;
a surgical tool configured to resect a first portion of a tumor including traversing the lumen defined by the cannula to access a tumor locus, the tumor locus located within a body of an imaging subject;
an actuator configured to position the cannula and the surgical tool according to a three dimensional coordinate system, the positioning including using information obtained from imaging a region within the imaging subject to identify the tumor locus in the three dimensional coordinate system;
wherein the surgical tool and the actuator are compatible with an imaging system and operable within or nearby the imaging system;
wherein the surgical tool is configured to access the tumor locus by traversing a bony structure comprising cranium; and
wherein one or more of the cannula or the surgical tool are configured to inhibit or suppress leakage of cerebrospinal fluid (CSF) during a surgical procedure.
